(19) United States
(12) Patent Application Publication
(10) Pub. No.: US 2021/0361222 A1
(43) Pub. Date: Nov. 25, 2021

(54) NEUROFEEDBACK DEVICES, SYSTEMS, AND METHODS FOR PAIN MANAGEMENT

(71) Applicants: Duke University, Durham, NC (US); East Carolina University, Greenville, NC (US)

(72) Inventors: Eric Elbogen, Durham, NC (US); Sara Rathman Battles, Aurora, OH (US); Amber Kathleen Alsobrooks, Durham, NC (US); Kiera Grace Molloy, Charleston, NC (US); Carmen Vincent Russoniello, Chocowinity, NC (US)

(21) Appl. No.: 17/327,640

(22) Filed: May 21, 2021

Related U.S. Application Data
(60) Provisional application No. 63/028,263, filed on May 21, 2020.

(51) Int. Cl. A61B 5/375; A61B 5/00; A61B 5/374; A61M 21/02

(52) U.S. Cl. CPC A61B 5/375; A61B 5/6803; A61M 2021/0022; A61B 5/0006; A61M 21/02; A61B 5/374

(57) ABSTRACT

Neurofeedback devices, systems, and methods for pain management. In some embodiments, the system includes one or more headset configured for detecting electrical activity of a brain of the subject; one or more electronic device in electronic communication with the one or more headset, the one or more electronic device comprising one or more processors configured to operate one or more software applications, which, when operated by the one or more processors, configure the one or more electronic device to perform the following functions: receive and analyze electrical activity data of the subject's brain from the one or more headset; and transmit an output to the subject, the output having a type selected by the subject, via a sound or visual output device based on an analysis of the received electrical activity data of the subject's brain; wherein the output is configured to reduce or relieve pain in the subject.

### CROSS-REFERENCE TO RELATED APPLICATIONS

The present application claims the benefit of U.S. Provisional Patent Application Ser. No. 63/028,263, filed May 21, 2020, the disclosure of which is incorporated herein by reference in its entirety.

### GOVERNMENT INTEREST

This invention was made with Government support under Federal Grant No. W81XWH-17-1-0591 awarded by the Department of Defense. The Federal Government has certain rights in the invention.

### TECHNICAL FIELD

The subject matter disclosed herein relates generally to devices used for electroencephalography. More particularly, the subject matter disclosed herein relates to a system, including a mobile software application and accompanying headset, for electroencephalography.

### BACKGROUND

For patients with traumatic brain injuries (TBI) and/or post-traumatic stress disorder (PTSD), chronic pain is a common side-effect of these trauma. Electroencephalograph (EEG) biofeedback, also known as “neurofeedback”, has been associated with lower pain but requires frequent travel to a clinic. Neurofeedback trains people using operant conditioning principles to gain more control over brain activity by balancing sympathetic and parasympathetic activity in the automatic nervous system to achieve levels of target brain activity for calmness and mental relaxation. Training that rewards select EEG frequencies increases or decreases of amplitudes (power) of those EEG frequencies, resulting in enduring modifications of brain activity and associated cognitive, emotional, and behavioral functions. EEG studies have shown that activity in pain perception pathways is linked to oscillations in alpha (8-13 Hz) waves of the brain, specifically suppressed alpha power and resting state peak alpha frequency. Suppression of alpha rhythm (alpha event-related desynchronization) can “open the gates” to increased pain input from the periphery. The stronger the magnitude of alpha event-related desynchronization in anticipation of pain, the greater the subjectively rated experience of pain.

Alpha neurofeedback training involves a person learning to identify and improve alpha synchrony, often in conjunction with adjunctive techniques, that is, meditation, music, or games. When a person's alpha synchrony closes “the gates,” increasing power in that band, the experience of pain decreases. According to the Association for Applied Psychophysiology's clinical efficacy classification system, neurofeedback is efficacious or probably efficacious in ameliorating symptoms of pain from various physical and psychological origins as well as when pain is comorbid with conditions such as PTSD. Changes in brainwave bandwidth activity, specifically increases in alpha power, have been demonstrated among individuals with chronic pain who undergo neurofeedback. Neurofeedback has successfully treated pain in complex regional pain syndrome (CRPS) type 1, fibromyalgia, spinal cord injuries, and trigeminal neuralgia. Neurofeedback has been associated with decreased pain and fatigue, improvements in worst pain, and decreased pain intensity.

Effectively treating chronic pain is critical, but existing treatments pose barriers. Pharmacological approaches like opioids and other narcotic treatments elevate risk of abuse and harmful side effects. Non-pharmacological treatments such as Cognitive Behavioral Therapy can be safe and effective in treating pain, but patients do not always optimally utilize this type of treatment.

Thus, there is a need for neurofeedback systems and methods for pain management for patients that are both effective and viable for patients with chronic pain, PTSD, and/or TBI.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

Provided herein are systems and methods for reducing pain in a subject using neurofeedback. In one aspect, a system for reducing pain in a subject is provided, the system comprising: one or more headset configured for detecting electrical activity of a brain of the subject; one or more electronic device in electronic communication with the one or more headset, the one or more electronic device comprising one or more processors configured to operate one or more software applications, which, when operated by the one or more processors, configure the one or more electronic device to perform the following functions: receive and analyze electrical activity data of the subjects brain from the one or more headset; and transmit an output to the subject, the output having a type selected by the subject, via an output electronic device, based on an analysis of the received electrical activity data of the subjects brain, where the output electronic device is the one or more electronic device or another device in communication with the one or more electronic device; wherein the output is configured to reduce or relieve pain in the subject.

In some embodiments, the one or more headset comprises an electroencephalography (EEG) headset or headband. In some embodiments, the one or more headset is configured for detecting and continuously measuring EEG power or amplitude of brain waves of the subject. In some embodiments, the brain waves of the subject include brain waves selected from the group consisting of alpha waves, delta waves, theta waves, beta waves, gamma waves, and combinations thereof. In some embodiments, the one or more headset and one or more electronic device are in wired or wireless electronic communication with each other. In some embodiments, the wireless electronic communication comprises BLUETOOTH®, Wi-Fi, 3G, LTE, 5G, or a wireless data communications network.

In some further embodiments, the one or more electronic device is selected from the group consisting of: a mobile phone, a smart phone, a tablet, a tablet personal computer (PC), a PC, a server, a workstation, and a computer. In some embodiments, the one or more electronic device is configured by the software application to display a graphical user interface (GUI) to the subject, wherein the one or more electronic device receives input from the subject via the GUI. In some embodiments, the type of output comprises visual output, sound output, vibration output, or haptic output. In some embodiments, analysis of the received electrical activity data comprises determining a baseline brain wave threshold by calculating an average brain wave power or amplitude value of the received electrical activity data during a baseline time interval. In some embodiments, after the baseline time interval has concluded, the one or more electronic device is configured to continue receiving electrical activity data of the subject's brain; wherein the average brain wave power or amplitude is an average alpha wave power or amplitude and the baseline brain wave threshold is a baseline alpha wave threshold.

In some further embodiments, in response to the one or more electronic device receiving, after the baseline time interval, electrical activity data having an alpha power or amplitude greater than the baseline alpha wave threshold, the one or more electronic device is configured to transmit the output with an intensity set to a level indicating that the subject is acting in a manner that is consistent with a calmer brain state. In some embodiments, in response to the one or more electronic device receiving, after the baseline time interval, electrical activity data having an alpha power or amplitude lower than the baseline alpha wave threshold, the one or more electronic device is configured to transmit the output with an intensity set to a level indicating that the subject is acting in a manner that is not consistent with a calmer brain state.

In another aspect, a method for reducing pain in a subject is provided, the method comprising: detecting, using a headset, electrical activity of the brain of the subject; receiving, at one or more electronic device, electrical activity data of the subject's brain from the one or more headset; analyzing, at the one or more electronic device, the received electrical activity data of the subject's brain; transmitting an output to the subject, via an output electronic device, based on a result of analyzing the received electrical activity data of the subject's brain, where the output electronic device is the one or more electronic device or another device in communication with the one or more electronic device; wherein the output is configured to reduce or relieve pain in the subject.

In some embodiments, the one or more headset comprises an electroencephalography (EEG) headset or headband. In some embodiments, the method further comprises continuously measuring EEG power or amplitude of brain waves of the subject while the headset is on the subject and powered on. In some embodiments, the brain waves of the subject include brain waves selected from the group consisting of: alpha waves, delta waves, theta waves, beta waves, and/or gamma waves. In some embodiments, the one or more electronic device is selected from the group consisting of: a mobile phone, a smart phone, a tablet, a tablet personal computer (PC), a PC, a server, a workstation, and a computer. In some embodiments the method further comprises displaying a graphical user interface (GUI) to the subject; and receiving input from the subject via the GUI.

In some embodiments, the output comprises visual output, sound output, vibration output, or haptic output. In some embodiments, analyzing the received electrical activity data comprises determining a baseline brain wave threshold by calculating an average brain wave power or amplitude value of the received electrical activity data during a baseline time interval. In some embodiments, after the baseline time interval has concluded, the one or more electronic device is configured to continue receiving electrical activity data of the subject's brain; wherein the average brain wave power or amplitude is an average alpha wave power or amplitude and the baseline brain wave threshold is a baseline alpha wave threshold.

In some embodiments, the method further comprises responsive to the one or more electronic device receiving, after the baseline time interval, electrical activity data having an alpha power or amplitude greater than the baseline alpha wave threshold, the method further comprises transmitting, by the one or more electronic device, the output with an intensity set to a level indicating that the subject is acting in a manner that is consistent with a calmer brain state. In some embodiments, the method further comprises responsive to the one or more electronic device receiving, after the baseline time interval, electrical activity data having an alpha power or amplitude lower than the baseline alpha wave threshold, the method further comprises transmitting, by the one or more electronic device, the output with an intensity set to a level indicating that the subject is acting in a manner that is not consistent with a calmer brain state.

In yet another aspect, an electronic device comprising one or more processors configured to operate one or more software applications is provided, which, when the software applications is operated by the one or more processors, configure the one or more electronic device to perform the following functions: receive electrical activity data corresponding to brain electrical activity of a subject; and provide or transmit an output to the subject at an intensity, the intensity being based on an analysis of the received electrical activity data of the subject's brain; or a control signal to an output electronic device to provide an output to the subject at an intensity based on an analysis of the received electrical activity data of the subject's brain; wherein the output is configured to reduce or relieve pain in the subject.

In another aspect, a method for reducing pain in a subject is provided, the method comprising: receiving electrical activity data corresponding to brain electrical activity of a subject; and providing or transmitting: an output to the subject at an intensity, where the intensity is based on an analysis of the received electrical activity data of the subject's brain; or a control signal to an output electronic device to provide an output to the subject at an intensity based on an analysis of the received electrical activity data of the subject's brain; wherein the output is configured to reduce or relieve pain in the subject.

Although some of the aspects of the subject matter disclosed herein have been stated hereinabove, and which are achieved in whole or in part by the presently disclosed subject matter, other aspects will become evident as the description proceeds when taken in connection with the accompanying drawings as best described hereinbelow.

### BRIEF DESCRIPTION OF THE FIGURES

The presently disclosed subject matter can be better understood by referring to the following figures. The components in the figures are not necessarily to scale, emphasis instead being placed upon illustrating the principles of the presently disclosed subject matter (often schematically). In the figures, like reference numerals designate corresponding parts throughout the different views. A further understanding of the presently disclosed subject matter can be obtained by reference to an embodiment set forth in the illustrations of the accompanying drawings. Although the illustrated embodiment is merely exemplary of systems for carrying out the presently disclosed subject matter, both the organization and method of operation of the presently disclosed subject matter, in general, together with further objectives and advantages thereof, may be more easily understood by reference to the drawings and the following description. The drawings are not intended to limit the scope of this presently disclosed subject matter, which is set forth with particularity in the claims as appended or as subsequently amended, but merely to clarify and exemplify the presently disclosed subject matter.

For a more complete understanding of the presently disclosed subject matter, reference is now made to the following drawings in which

FIG. 1A illustrates a subject utilizing an example system according to some embodiments of the present disclosure;

FIG. 1B illustrates an example headset and example electronic device in communication with each other making up the system according to some embodiments of the present disclosure;

FIG. 2A, FIG. 2B, and FIG. 2C illustrate several example EEG headsets that can be used in the system of some of the embodiments according to the present disclosure;

FIG. 3A, FIG. 3B, and FIG. 3C illustrate several example electronic devices that can be used in the system of some of the embodiments according to the present disclosure;

FIG. 4A, FIG. 4B, FIG. 4C, FIG. 4D, and FIG. 4E illustrate several pages displayed on a graphical user interface operated by a software application according to some embodiments of the present disclosure;

FIG. 5A and FIG. 5B is a flow chart describing steps of a method according to some embodiments of the present disclosure; and

FIG. 6 illustrates graphical representations of different types of brain waves in humans.

### DETAILED DESCRIPTION

The disclosure hereinbelow provides systems, devices, and methods for reducing and/or eliminating chronic pain. Systems and methods disclosed herein can be used in hospitals (e.g., inpatient settings) and with populations in other environments (e.g., at a residence, outpatient facility, clinic, etc.). The disclosed systems and methods are particularly useful for individuals suffering from chronic pain, traumatic brain injury (TBI), post-traumatic stress disorder (PTSD), and other traumas causing the subject or patient chronic pain. The subject matter provided herein includes a system comprising an EEG headband or headset configured to detect electrical activity of the brain of a subject and transmit electrical activity data to an electronic device for analysis. The electronic device is then configured to provide neurofeedback to the subjects based on the analysis of the electrical activity data of the subject's brain.

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

The term “effective amount” or “therapeutically effective amount” refers to an amount sufficient to effect beneficial or desirable biological and/or clinical results.

As used herein, the term “subject” and “patient” are used interchangeably. In some embodiments, the subject comprises a human who is undergoing neurofeedback therapy with a device as prescribed herein.

Unless otherwise defined, all technical terms used herein have the same meaning as commonly understood by one of ordinary skill in the art to which this disclosure belongs.

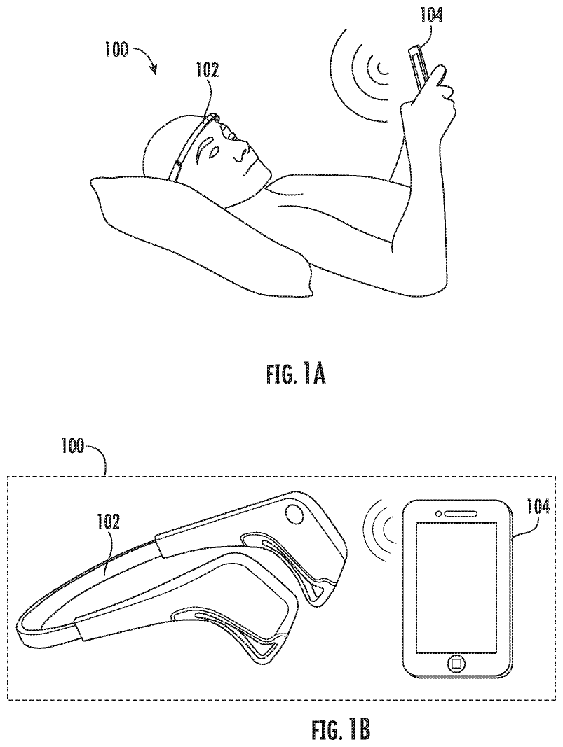

Reference is now made to the several figures included with this application, the first of which is FIG. 1A, which illustrates an example neurofeedback therapy system 100 according to some embodiments of the present disclosure. The system 100 is configured to provide neurofeedback to a subject S by analyzing the subject's brain electrical activity and providing output to the subject S, the output being configured for helping the subject reduce chronic pain. In some embodiments, the system 100 comprises an EEG headset 102 in electronic communication with an electronic device 104. The headset 102 is to be worn by a subject S to detect and measure electrical activity of a brain of the subject S. The system 100 can be used by the subject S to help reduce chronic pain associated with a TBI, PTSD, or other trauma experienced by the subject S. The system 100 can also be used to help the subject S treat chronic pain associated with any disease, disorder, or other injury developed before, during, or after birth.

As illustrated in FIG. 1B, the electronic device 104 is in electronic communication with the EEG headset 102. In some embodiments, the electronic communication can be facilitated via a wired or wireless connection between the EEG headset 102 and the electronic device 104. In some embodiments, the wireless connection can be via BLUETOOTH®, Wi-Fi, 3G, LTE, 5G, or other wireless data network. In some embodiments, the wired connection can be via USB, Ethernet, HDMI, or any other suitable wired connection.

As illustrated in FIG. 1A, a subject S can lay in a relaxed state while participating in the neurofeedback therapy session to help reduce their chronic pain. To start the session, in some embodiments, the subject S will turn on and adjust the EEG headband 102 to ensure a proper fit and measurement of brain electrical activity. The subject S can lay or sit or stand in any relaxed position they wish. The subject S is laying in a relaxed position, supported by several pillows. The subject S can then turn on or activate/unlock the electronic device 104 and ensure that it is connected, via wired or wireless communication means, to the EEG headband 102. For example, and without limitation, the electronic device 104 can be in electronic communication with the EEG headband 102 via BLUETOOTH®. In such an embodiment, the electronic device 104 will need to have BLUETOOTH® enabled and sync to the EEG headband 102. Although not illustrated in FIG. 1A or FIG. 1B, the electronic device 104 can comprise one or more electronic devices. For instance, the EEG headband 102 can be connected to one or multiple electronic devices via Wi-Fi, BLUETOOTH®, etc.

In some aspects, the electronic device 104 comprises one or more processors, one or more computer readable media, and executable instructions that, when executed, cause the electronic device 104 to operate a software application, mobile application, “app”, or other piece of software or program. In some embodiments, the software application can generate or cause to be displayed a graphical user interface (GUI) on a screen. The screen can be a screen on the electronic device or a screen in communication with the electronic device such as, for example and without limitation, a display, a monitor, a touch screen, or other suitable display. The software application further includes an algorithm that is configured to receive and processes brain activity and provides feedback according to the measured output based on the thresholds described hereinbelow. In some embodiments, the software application is configured to train users to specifically increase their EEG alpha brain waves (alpha power), which has been shown in research literature to be associated with reduced pain intensity.

In some embodiments, the software application can be operated by the electronic device 104 or in the EEG headband 102, or it can be operated by a standalone device such as a server, PC, cloud server, or other computing device. If the software application is not operated on a smart phone or other device that the subject S can easily manipulate, the subject S is provided with some device or apparatus giving them access to start and run the software application and receive output from the software application. The disclosure herein details embodiments where the subject S is provided with a smart phone or other handheld electronic device which has a touch screen to operate a GUI controlled by the software application, but those having ordinary skill in the art will appreciate that any suitable computing device can be provided to the subject S to operate the software application run on another computing device located anywhere in the world.

The subject S can select the app on the electronic device 104 and start the neurofeedback therapy session, which is operated by the software app and the EEG headband 102. In some aspects, the subject S starts the software application (i.e., by clicking, selecting, or otherwise indicating to the electronic device that the software application should start) and the neurofeedback therapy session will begin.

After the software application has started operating, the GUI appears on the screen associated with the electronic device 104 and the software application starts receiving electronic signals from the EEG headband 102, when the electronic device 104 and the software application are in electronic communication with the EEG headband 102. The EEG headband 102 is configured to detect and measure brain electrical activity of the subject S. For example and without limitation, the EEG headband 102 is configured to detect and measure alpha waves, delta waves, theta waves, beta waves, and/or gamma waves from the subject S. Those having ordinary skill in the art will appreciate that alpha waves from the subject's brain indicate quietly flowing thoughts and that the subject S is in a meditative state (i.e., calmness). In some embodiments, this alpha wave state is the desired state for a neurofeedback procedure in accordance with the presently disclosed subject matter.

Delta waves indicate dreamless sleep and deepest meditation for a subject S. A subject S producing brain wave activity consistent with delta waves stimulate healing in the body of the subject S. Theta waves indicate deep meditation, another highly desired state. Beta waves indicate a normal waking state of consciousness, consistent with average levels of attention and concentration. Finally, gamma waves indicate that the subject S is in the highest state of attention. In some embodiments, the EEG headset 102 can detect the other types of brain waves beyond alpha waves, such as, for example and without limitation, beta, gamma, delta, and theta waves. In some embodiments, the system 100 focuses on the alpha waves for the procedures described herein.

Once the software application has been started and is running, and the electronic device 104 is in electronic communication with the EEG headband 102, the electronic device 104 and the software application are configured to receive brain electrical activity (i.e., brainwave activity) from the EEG headband 102. In some embodiments, the EEG headband 102 is configured to continuously measure EEG power or amplitude of the brain waves and transmit brain electrical activity data, including the EEG power or amplitude of the detected brain waves to the software application. The EEG headband 102 can transmit the brain electrical activity data periodically (i.e., once every period of time), substantially real-time (i.e., as the measurement occurs), or by any other means suitable for transmission and reception of the brain electrical activity data.

In some embodiments, the software application GUI offers various menus to the subject S giving output to the subject S and requesting inputs from the subject S. For example and without limitation, in some embodiments, before any therapy is provided to the subject S, but after the EEG headset 102 and the electronic device 104 are connected and communicating, the subject S can be provided a menu to select one of multiple sounds or visuals to choose from in order to help calm the subject S. The subject S can also select and provide various other inputs to the GUI, including providing input to start the neurofeedback session, providing pain, anger, and anxiety levels (i.e., on a scale of 0 to 10, 0 to 100, or any other suitable scale), and other inputs as well.

In some embodiments, the software application running on the electronic device 104 is configured to receive and analyze the brain activity data from the EEG headset 102 worn by the subject S. As described further hereinbelow, the electronic device 104 and the software application are configured to process the brain electrical activity and then transmit the audio or visual output to the subject S to help calm them and reduce their chronic pain. In some embodiments, the software application and electrical device can provide audio output, visual output, a light signal output, haptic feedback output, vibrations or any other suitable output.

After the subject S has provided their various inputs and started the session, the GUI, in some embodiments, can instruct the subject S to get into a comfortable position and to ensure that the electronic device 104 has a good connection with the EEG headband 102. In some aspects, if the EEG headband 102 has a poor quality signal with the electronic device 104 for greater than a few seconds, for example and without limitation, about 5 seconds, the neurofeedback therapy sessions will halt and the GUI will prompt the subject S to adjust the EEG headset 104 until a good signal quality is re-attained.

Next, the system 100 is configured to determine or establish a baseline brain wave threshold to be used to determine when and how output is sent to the subject S. Prior to calculating this threshold, in some embodiments, the subject S is instructed, via the GUI to close their eyes for one minute while the electronic device 104 and software application analyze the brain wave power or amplitude of the received brain electrical activity data. The electronic device 104 and software application are configured to analyze the brain electrical activity data and determine the threshold value by calculating an average brain wave power or amplitude value during the one-minute interval while the subject S has their eyes closed. This calculated average is the baseline brain wave threshold value. In some cases, the subject S may have multiple therapy sessions. Each time they start a new therapy session, their baseline brain wave threshold can be re-calculated as described above. In some embodiments, the brain wave threshold can be determined based on the average power or amplitude of one or more different types of brain waves, including, for example, alpha, beta, theta, gamma, or delta waves. For example and without limitation, the system 100 is configured to determine or establish a baseline alpha wave threshold to be used to determine when and how output is sent to the subject S. Prior to calculating this threshold, in some embodiments, the subject S is instructed, via the GUI to close their eyes for one minute while the electronic device 104 and software application analyze the alpha wave power or amplitude of the received brain electrical activity data. The electronic device 104 and software application are configured to analyze the brain electrical activity data and determine the threshold value by calculating an average alpha wave power or amplitude value during the one-minute interval while the subject S has their eyes closed. This calculated average is the baseline alpha wave threshold value. In some cases, the subject S may have multiple therapy sessions. Each time they start a new therapy session, their baseline alpha wave threshold can be re-calculated as described above. The same process can be repeated for any individual brain wave data or data on any combination of different brain waves.

Once the one-minute baseline time interval has concluded, the electronic device 104 is configured to start the neurofeedback therapy session. In some embodiments, the neurofeedback therapy session can last for any given period of time. For example and without limitation, the therapy session can last about 1, 5, 10, 15, 20, 30 or more minutes. In some embodiments in particular, the therapy sessions can last about 10 minutes after the baseline time interval has concluded.

During the therapy session, the EEG headset 102 is configured to continuously detect and monitor the brain electrical activity of the subject S as well as transmit the brain electrical activity data to the electronic device 104. As the electronic device 104 receives the brain electrical activity data from the EEG headset 102, the electronic device 104 is configured to analyze the received activity data and if the alpha wave amplitude or power of the data is above the threshold, the electronic device 104 is configured to transmit an output to the subject S indicating that the subject S should continue doing whatever they are doing currently with respect to breathing, thinking, feeling, muscle tension, body posture, or mental visualization because their current actions, thoughts, and posture are consistent with a calmer brain state. If the received activity data indicates that the alpha wave amplitude or power is below the threshold, the electronic device 104 is configured to transmit an output to the subject S that the subject S should try to change body posture, relax muscle tension, breath more naturally, and think, feel, or visualize something different Because the power or amplitude of the alpha brain waves is continuously changing, depending on the stimulus' effect on the EEG headset 102, the detection, analysis, and other procedures described herein are performed substantially in real-time. In other words, as the alpha brain waves amplitude changes, the EEG headset 102 detects the changes and transmits the data to the electronic device 104 for analysis, all performed substantially in real-time. The brain wave data is detected and transmitted by the EEG headset 102 as quickly as the processors onboard the headset 102 will allow and the transmission speed to the electronic device 104 is based on wireless communication method and electronic device 104 throughput parameters. For example and without limitation, the EEG headset 102 can detect brain waves (e.g., alpha waves) ten or more times per second. For example, the EEG headset 102 can detect brain waves (e.g., alpha waves) 10, 15, 20, 25, 30, 35, 40, 45, 50, 55, 60, or 100 times or more per second. In some embodiments, the EEG headset 104 can be configured to transmit the detected data in substantially real-time (i.e., 10 time per second or any of the other frequencies mentioned above) based on wireless communication throughput parameters.

According to the present disclosure, the subject S can take a number of approaches to achieve a relaxed state. Because the mechanism for relaxation can be very individualized, the software application does not necessarily tell the subject what particular actions to take. The software application provides feedback or an output to the subject S to support or deter a particular activity that is increasing or decreasing their alpha wave power. For example, if performing deep breathing causes a subject S to produce higher alpha power, the output provided by the software application increases in intensity or maintains a comfortable intensity. Conversely, if deep breathing does not cause alpha waves to reach or stay above a prescribed threshold, the output provided by the software application decreases in intensity, encouraging the subject S to try a different approach. Alternatively, as described herein, the output can change in intensity differently depending on how the software application is designed as long as the instructions to the subject S on what those changes mean are clear. For example, a decrease in intensity of the output could mean “continue what you're doing” and an increase in intensity of the output could mean “change your body posture,” or the like.

Furthermore, in some embodiments, the system 100, via the electronic device 104 and software application, is configured to alter the intensity of the selected output with a buffer such that the intensity is not changed too frequently. For example and without limitation, the electronic device 104 can be configured to have a delay of about 5 seconds before altering the intensity of the output. In some other embodiments, the delay can be 1, 2, 5, 8, 10, seconds or any other suitable delay. The delay is the time in which the electronic device 104 will cause the output intensity to change if the alpha wave power is above or below the certain threshold. In other words, if the alpha wave power is not above or below the threshold for the delay time, the output intensity is not changed by the electronic device 104.

In addition, in order to obtain the most accurate brain wave data from the EEG headset 102, in some embodiments, the alpha wave power value used for determining the threshold or the current alpha wave power can be obtained by averaging the alpha power value from each of the one or more headset sensors. If one sensor is disconnected, rather than give a false “below threshold” signal/output or alerting the subject S to the disconnected sensor immediately, the output remains the same unless the sensor(s) remained disconnected for greater than about 5 seconds (or some other specified time). Moreover, if a subject's alpha wave power or amplitude value is hovering at the threshold, they may be above or below threshold for milliseconds at a time. Rather than changing the intensity of the output in a way that could be distracting, stressful, or confusing for the subject S, the electronic device 104 and software application are configured to alter the intensity of the output only if the subject changed to above or below the threshold for at least a given time, such as for example, 5 seconds (or any other specified time). The intensity (i.e., volume, brightness, vibration intensity, etc.) of the output remains the same if the alpha wave power of a subject rapidly switches above and below the threshold. In clinical EEG, Power Spectral Density (a derivative of raw EEG expressed as the average of the power over a specified period of time) is often used to smooth EEG signals. In some embodiments of the present disclosure, the electronic device 104 and software application can be configured to calculate power spectral density from the raw EEG signals detected by the EEG headset 102.

In some embodiments, the electronic device 104 is configured to transmit an audio output, that increases in volume or intensity, to the subject S as the power of the received alpha wave data is above the threshold. If the power of the received alpha wave power or amplitude dips below the threshold, the electronic device 104 is configured to transmit an audio output that decreases in volume or intensity or is lower in volume or intensity than the volume of the audio output is when the alpha wave power or amplitude is above the threshold. In some embodiments, the opposite can be true. For example, instead of increasing the volume or intensity of the audio output when the alpha wave amplitude is above the threshold, the volume or intensity of the audio output can be decreased. Similarly, instead of decreasing the volume or intensity of the audio output when the alpha wave amplitude is below the threshold, the volume or intensity of the audio output can be increased. The subject S is informed of what the various outputs mean and what an increased volume and a decreased volume mean in terms of what the subject S should do to calm their brain.

In some embodiments, the audio output can come from the electronic device 104 itself, for example, from speakers on the electronic device 104. In some other embodiments, the electronic device 104 can transmit a signal to a speaker device or other device configured to convey the audio sound to the subject S. In another embodiment, for example, the electronic device 104 can be configured to transmit a signal wireless or wired headphones in the ears of the subject S to play the audio output at the configured volume or intensity based on the current amplitude of the alpha waves being above or below the threshold. The type of audio output can be the audio output selected by the subject S on the GUI. For example, before the procedure starts, the GUI can query the subject S for a selection on a type or option of sounds (i.e., the sound of waves, music, nature sounds, etc.) and the subject S can select one of the options and the audio output will be the sound selected by the subject S.

Another output to the subject S can be visual or haptic or vibration output. For example and without limitation, the electronic device 104 can output a visual display to the subject S via a display on the electronic device 104 itself or any other suitable display, screen, monitor, television, or other display that the electronic device 104 is in electronic communication with. In some embodiments, the output of the visual display change in various ways to indicate to the subject S that their current alpha wave power or amplitude is above or below the threshold. For example and without limitation, the visual display output could be a calming picture for the subject S and the output can include increasing or decreasing the brightness of the calming picture based on whether the amplitude of the alpha waves of the subject S is above or below the threshold (i.e., increase brightness if above the threshold and decrease the brightness if below the threshold, or decrease brightness if above the threshold, and increase the brightness if below the threshold). Similar outputs can be given via haptic or vibration output. For example, the electronic device 104 itself can give haptic or vibration feedback or some other device that is touching the subject S can be triggered to give the output to the subject S based on signal transmissions from the electronic device 104. In some embodiments, the subject S can select the type of output they wish to receive. For example, a deaf person cannot hear the audio output, so they would want to receive visual, haptic, or vibration output. The GUI can give the subject S the option to choose which of these types of outputs they wish to receive.

Continual reinforcement of the posture of the subject S, thought process, thinking, etc. by these outputs trains the subject S which mental, physical, and emotional states are associated with physiological relaxation.

In some embodiments, the electronic device 104 and software application continues to provide the selected output based on the power or amplitude of the subjects alpha waves detected during the session. Additionally, the software application and electronic device 104 can be configured to detect and save EEG alpha, beta, delta, gamma, and theta wave data as well as pain, anger, and stress data on the electronic device 104, in the cloud, on a server in communication with the electronic device 104 or on some other computer. The data can also be transmitted to an electronic device owned by the subject S, printed off, or otherwise saved or logged to one or more computer readable media for further analysis or viewing. In some embodiments, the file(s) holding the additional brain-wave and subject S pain, stress, and anger level data can be password-protected, encrypted, and de-identified. In some embodiments, access to the file can be made intentionally difficult to find on the electronic device 104 for privacy reasons.

In some embodiments, when the neurofeedback therapy session is completed, the GUI of the software application can prompt the subject S to rate their current level of pain, anger, and stress on a given scale, denoting their post-session status. The subject S is then provided, via the GUI, a score, or other indication, which denotes the number of seconds their EEG alpha wave levels were above the threshold. In some embodiments, the subject S can be provided with the number of completed sessions they have been through.

While the above disclosure has been described in a manner such that the brain electrical activity data is received by an electronic device 104 from an EEG headset 102, in some embodiments, the system 100 merely comprises the electronic device 104 with the software application in communication with a computing system or some other device where the electronic device 104 receives the brain electrical activity data from the computing system or other device and performs the same functions as described above. For example, the electronic device 104 can receive the brain electrical activity data from a cloud server, a server on a local area network (LAN) where the electronic device is connected, or any other suitable computing device. Furthermore, the electronic device 104 can receive the brain electrical activity via taking pictures of a hard copy of a graph of the activity data. Those having ordinary skill in the art will appreciate that the electronic device 104 and software application performing the functions described herein can produce the same functions described herein while receiving the brain electrical activity data via any suitable means. The electronic device 104 and software application need only receive and analyze the data—it does not matter from what or whom the data is directly received from.

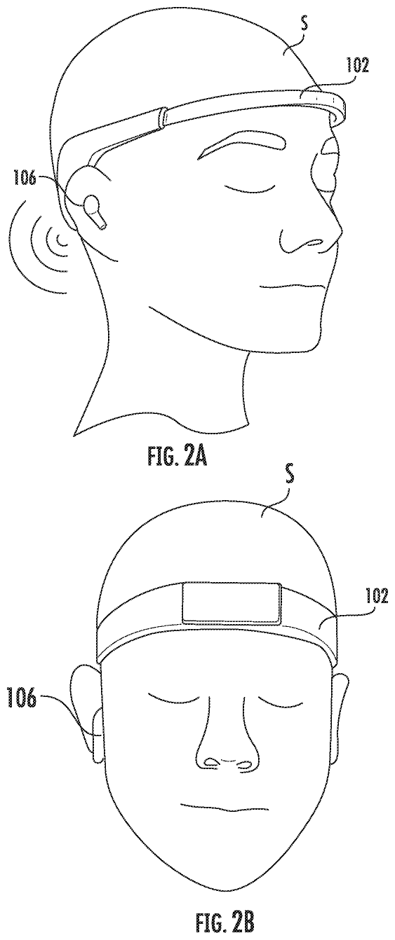

FIG. 2A to FIG. 2C illustrate various types of EEG headbands 102 which can be used for the neurotherapy sessions according to some embodiments of the present disclosure. In some embodiments, as described above, the subject S can be provided with one or more wireless or wired headphones 106 to receive audio output according to the embodiments above. In some embodiments, the EEG headband 102 can be a conventionally available device or a customized device in accordance with the present disclosure.

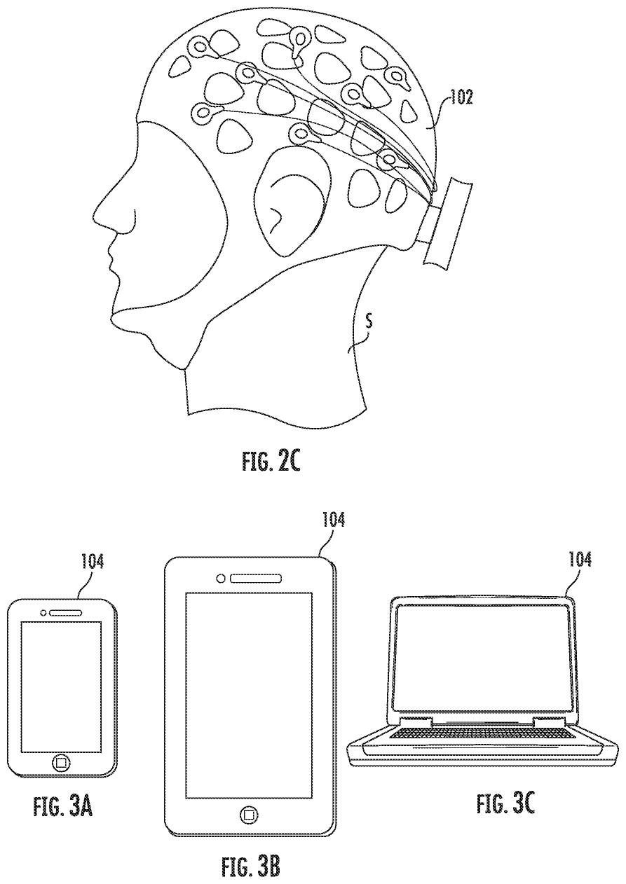

FIG. 3A to FIG. 3C illustrate various types of electronic devices 104 that can be used as part of the system 100 according to various embodiments of the present disclosure. In some embodiments of the present disclosure, the electronic device 104 can comprise any suitable computing device that comprises one or more processors configured for executing one or more software instructions, including operating the software application, one or more computer readable media for storing the software application, as well as any other software necessary to operate the electronic device 104. For example, and without limitation, the electronic device 104 can comprise a mobile phone, smartphone, iPod Touch®, personal/portable electronic device, or any other suitable handheld device like that shown in FIG. 3A. In some other embodiments, the electronic device 104 can comprise a tablet PC, a tablet device, an iPad®, or any other suitable tablet-like device like that shown in FIG. 3B. In some other embodiments, the electronic device 104 can comprise a laptop, a computer, a personal computer, a server, a cloud server, an iMac®, or any other suitable computer device that can connect to the EEG headset 102 and receive input from the subject S and provide output to the subject S as described above.

In some embodiments, the electronic device 104 can provide visual, audio, haptic, or vibration output to the subject S via a built-in screen or speakers shown in FIG. 3A through FIG. 3C as well as any circuitry (i.e., circuitry for haptic and vibration feedback) contained within the enclosure of the electronic device 104.

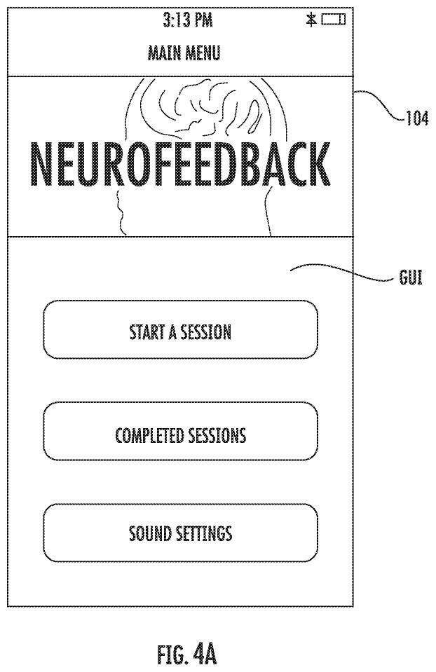

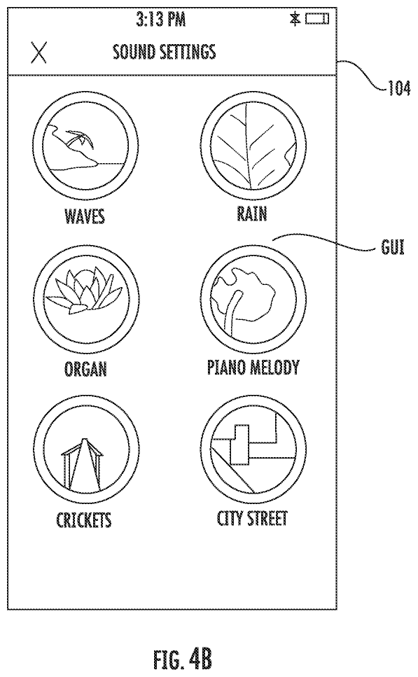

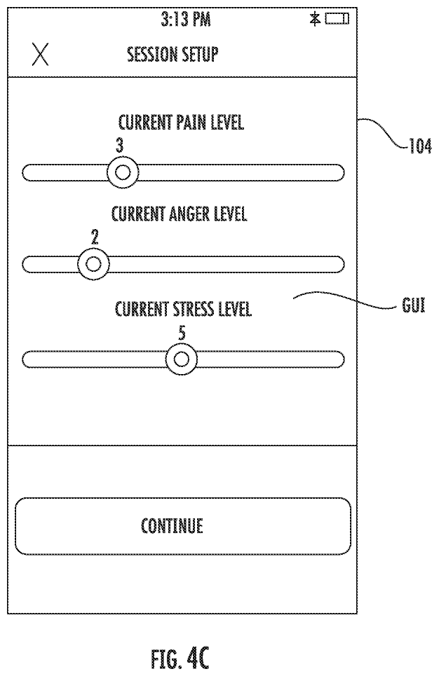

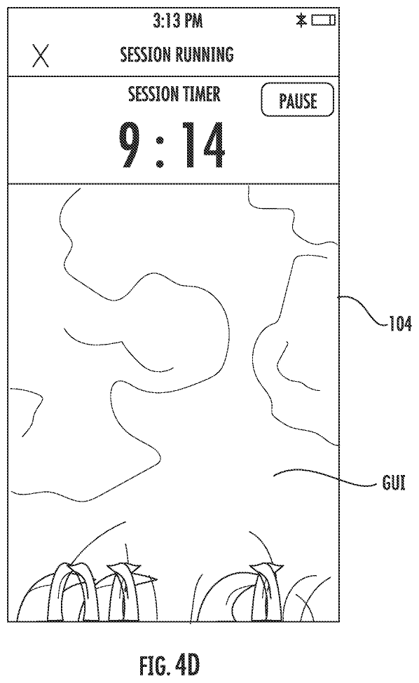

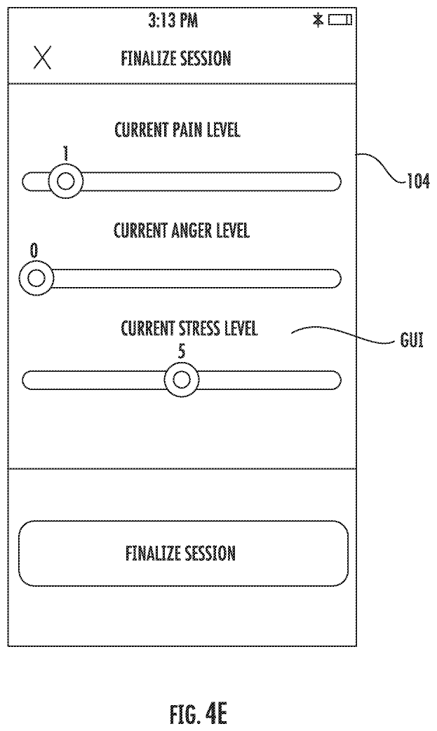

FIG. 4A through FIG. 4E illustrate various examples of an electronic device 104 displaying various pages of the GUI operated by the software application according to the present disclosure. In some embodiments, the GUI displays a series of “pages” each with various input and output possibilities based on the intended purpose of the particular page being displayed. For example and without limitation, when the software application is first started on the electronic device 104, the GUI can show, as a first page, a main menu like that shown in FIG. 4A, allowing the subject S to start a new neurofeedback session, review completed sessions, and adjust sound settings by selecting (e.g., using their finger or other selector) one of the corresponding buttons displayed. Instead of sound settings, lighting, vibration, and haptic settings can also be adjusted. For example, frequency, patterns, or intensity of vibrations or haptic feedback, etc. can be adjusted.

FIG. 4B illustrates an example page of the GUI associated with the software application if the “Sound Settings” button is selected from the main menu page shown in FIG. 4A. From this page of the software application, the subject S can select from any number of calming sounds to play during the neurofeedback session. Those having ordinary skill in the art will appreciate that any suitable sound can be displayed for the subject S to select from, not just those illustrated in FIG. 4B. Furthermore, those having ordinary skill in the art will appreciate that these sounds can be replaced by vibration settings, patterns, frequencies, haptic feedback settings, or visual display settings. For example and without limitation, the settings page could include a group of pictures for the subject S to select from for visual output during the neurofeedback session.

FIG. 4C illustrates an example page of the GUI displayed on the electronic device 104 if the “Start Session” button is selected from the main menu. In this illustration, the GUI provides slide bar selectors to the subject S to input their current levels of pain, anger, and stress prior to the start of the session. This particular page is provided for illustration purposes only, and should not be viewed as limiting, in any way, the display of these selectors for input by the subject S. For example and without limitation, the software application can provide an input text box where the subject S can directly enter their level numbers. Other methods of indicating the subject's current pain, anger, and stress levels are also envisioned, including any suitable way of indicating these levels to the software application.

FIG. 4D illustrates an example page of the GUI displayed on the electronic device 104 once the “CONTINUE” button is pressed from the previous page. The page of the GUI illustrated in FIG. 4D includes an example of what the GUI might display when the subject S is currently in the neurofeedback session. For example and without limitation, a session timer can be displayed with an ability to pause the session (i.e., the subject S selects the “PAUSE” button on the GUI). There can also be some indication that the neurofeedback session is running currently, such as, for example and without limitation, via a display of words stating that the session is running, or a red dot like when a smart phone camera is recording, or any other suitable indication.

Additionally, those having ordinary skill in the art will appreciate that, as described above, the output to the subject S can include displaying an image to help calm the mind of the subject S. At this page of the GUI, the output image or video could be displayed during the session. In some embodiments, a series of images or videos are displayed to the subject S. In some embodiments, at this page, the subject S could also be provided with additional sounds to select or different vibration or haptic feedback as well.

FIG. 4E illustrates an example page of the GUI displayed on the electronic device 104 once the neurofeedback session has ended. After the neurofeedback session has ended, the subject S can be prompted to provide new inputs on their current levels of pain, anger, and stress. As described above regarding FIG. 4C, the slide bar selectors can be replaced with any suitable variation of input text boxes or other input arrangement for the subject S. The subject S can then be prompted to finalize the session by tapping or selecting a corresponding button on the GUI.

In an example usage scenario, the application provides 10-minute neurofeedback sessions, with a goal of a subject completing 48 sessions in a 12-week intervention, approximately 4 sessions per week. One possible outcome of using the program is to establish subject habits such that the system is no longer needed, and the subject is able to self-direct, decreasing pain levels without the use of an EEG device.

The software application can optionally include additional features. These include, but are not limited to, a score based on number of seconds a subject had alpha levels above the threshold level during the neurofeedback session; a record tally tracking progress toward achieving a prescribed number of sessions; and other suitable features that will be evident to a person of skill in the art.

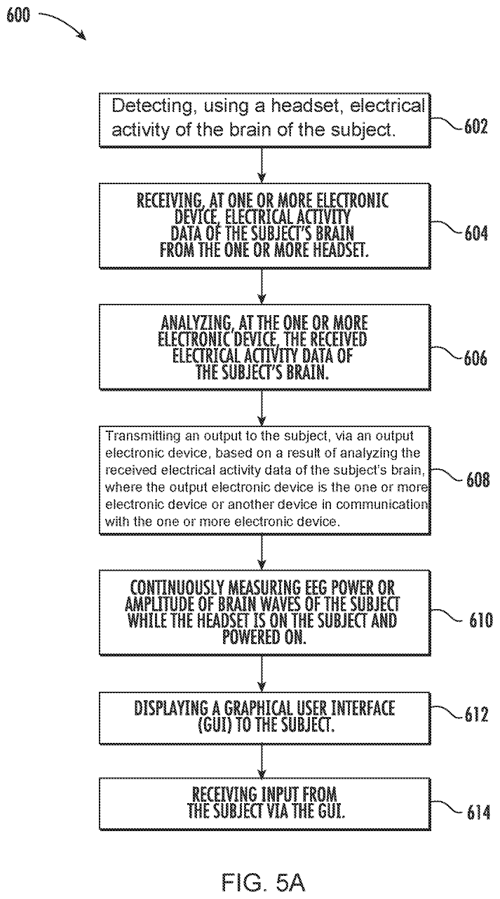

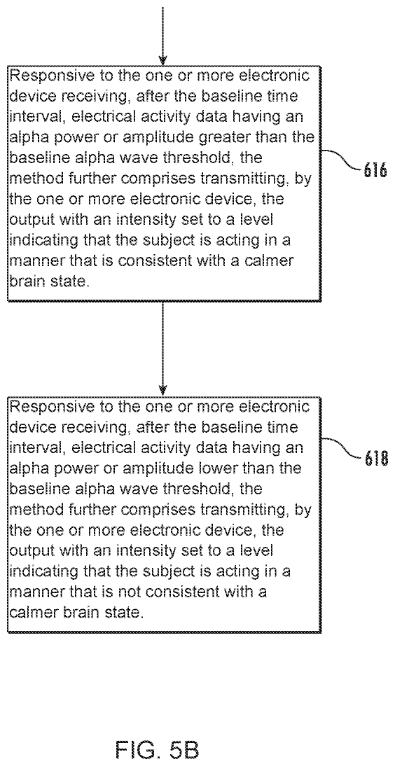

Another embodiment of the present disclosure provides a method of reducing chronic pain using the disclosed devices and methods. FIG. 5A and FIG. 5B illustrate various steps in a method 600 for reducing pain in a subject according to some embodiments of the present disclosure. In some embodiments, a first step 602 of the method 600 comprises detecting, using a headset, electrical activity of the brain of the subject. In some embodiments, a second step 604 of the method 600 comprises receiving, at one or more electronic device, electrical activity data of the subject's brain from the one or more headset. In some embodiments, a third step 606 of the method 600 comprises analyzing, at the one or more electronic device, the received electrical activity data of the subject's brain. In some embodiments, analyzing the received electrical activity data comprises determining a baseline alpha wave threshold by calculating an average alpha power or amplitude value of the received electrical activity data during a baseline time interval. In some embodiments, a fourth step 608 of the method 600 comprises transmitting, from the one or more electronic device, an output via a sound or visual output device based on a result of analyzing the received electrical activity data of the subjects brain; wherein the output is configured to reduce or relieve pain in the subject. In some embodiments, a fifth step 610 of the method 600 comprises continuously measuring EEG power or amplitude of brain waves of the subject while the headset is on the subject and powered on.

In some embodiments, a sixth step 612 of the method 600 comprises displaying a graphical user interface (GUI) to the subject. In some embodiments, a seventh step 614 of the method 600 comprises receiving input from the subject via the GUI. In some embodiments, a eighth step 616 of the method 600 comprises responsive to the one or more electronic device receiving, after the baseline time interval, electrical activity data having an alpha power or amplitude greater than the baseline alpha wave threshold, the method further comprises transmitting, by the one or more electronic device, the output with an intensity set to a level indicating that the subject is acting in a manner that is consistent with a calmer brain state. In some embodiments a ninth step 618 of the method 600 comprises responsive to the one or more electronic device receiving, after the baseline time interval, electrical activity data having an alpha power or amplitude lower than the baseline alpha wave threshold, the method further comprises transmitting, by the one or more electronic device, the output with an intensity set to a level indicating that the subject is acting in a manner that is not consistent with a calmer brain state. Any of the various steps in the method 600 above could be optional steps based on the particular setup of a system being used.

The software application can optionally adjust the brainwave analysis over time. This can be accomplished by applying one or more classification schemas that are set based on one or more machine learning algorithms trained on one or more training examples. In some embodiments, the average brain wave power or amplitude threshold (e.g., alpha brain wave power or amplitude threshold) can be adjusted or optimized or selected based on one or more machine learning algorithms trained on one or more training examples.

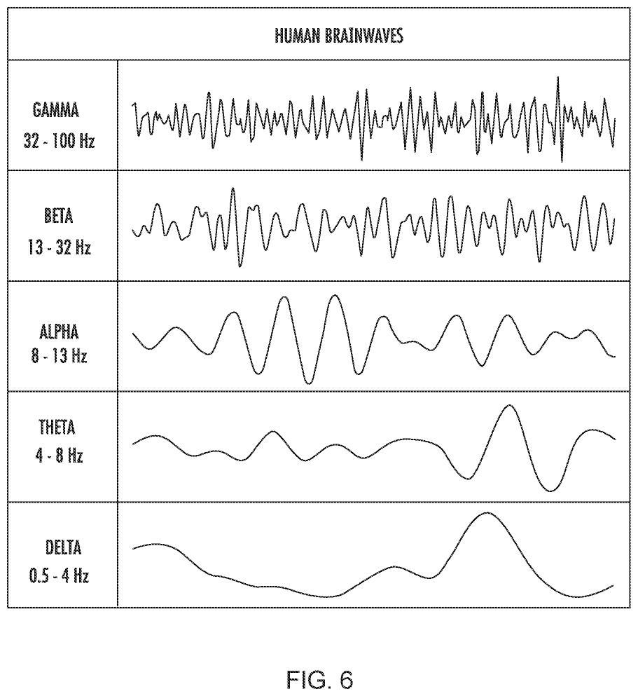

FIG. 6 illustrates example wave patterns of different brain waves in humans. As shown in the example, gamma brain waves have the highest frequency (i.e., 32-100 Hz) and are associated with the most alert attention of the brain. Delta waves have the lowest frequency (i.e., 0.5-4 Hz) and are associated with a very restful brain (e.g., deep, dreamless sleep).

As will be appreciated by one of skill in the art, the disclosed subject matter can be embodied as a method, a system, or a computer program product for implementing the functionality described herein. Accordingly, the present disclosure may take the form of an entirely hardware embodiment, an entirely software embodiment (including firmware, resident software, micro-code, app etc.) or an embodiment combining software and hardware aspects. Furthermore, the present disclosure may take the form of a computer program product on one or more non-transitory computer readable media having computer-usable program code embodied in the media. The user interface can likewise include any applicable format, such as software applications, input/output devices, touchscreens, display monitor, tablet, mobile devices, etc.

The subject matter disclosed herein can be implemented in software in combination with hardware and/or firmware. For example, the subject matter described herein can be implemented in software executed by a processor or processing unit. In one implementation, the subject matter described herein can be implemented using a computer readable medium having stored thereon computer executable instructions that when executed by a processor of a computer control the computer to perform steps. Example computer readable mediums suitable for implementing the subject matter described herein include non-transitory devices, such as disk memory devices, chip memory devices, programmable logic devices, and application specific integrated circuits. In addition, a computer readable medium that implements the subject matter described herein can be located on a single device or computing platform or can be distributed across multiple devices or computing platforms.

While at least one example embodiment of the present invention(s) is disclosed herein, it should be understood that modifications, substitutions and alternatives may be apparent to one of ordinary skill in the art and can be made without departing from the scope of this disclosure. This disclosure is intended to cover any adaptations or variations of the example embodiment(s). In addition, in this disclosure, the terms “comprise” or “comprising” do not exclude other elements or steps, the terms “a”, “an” or “one” do not exclude a plural number, and the term “or” means either or both. Furthermore, characteristics or steps which have been described may also be used in combination with other characteristics or steps and in any order unless the disclosure or context suggests otherwise. This disclosure hereby incorporates by reference the complete disclosure of any patent or application from which it claims benefit or priority.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The present subject matter can be embodied in other forms without departure from the spirit and essential characteristics thereof. The embodiments described therefore are to be considered in all respects as illustrative and not restrictive. Although the present subject matter has been described in terms of certain specific embodiments, other embodiments that are apparent to those of ordinary skill in the art are also within the scope of the present subject matter.

What is claimed is:

1. A system for reducing pain in a subject, the system comprising:
one or more headset configured for detecting electrical activity of a brain of the subject;
one or more electronic device in electronic communication with the one or more headset, the one or more electronic device comprising one or more processors configured to operate one or more software applications, which, when operated by the one or more processors, configure the one or more electronic device to perform the following functions:
receive and analyze electrical activity data of the subject's brain from the one or more headset; and
transmit an output to the subject, the output having a type selected by the subject, via an output electronic device, based on an analysis of the received electrical activity data of the subject's brain, where the output electronic device is the one or more electronic device or another device in communication with the one or more electronic device;
wherein the output is configured to reduce or relieve pain in the subject.

2. The system of claim 1, wherein the one or more headset comprises an electroencephalography (EEG) headset or headband.

3. The system of claim 1, wherein the one or more headset is configured for detecting and continuously measuring EEG power or amplitude of brain waves of the subject.

4. The system of claim 3, wherein the brain waves of the subject include brain waves selected from the group consisting of alpha waves, delta waves, theta waves, beta waves, gamma waves, and combinations thereof.

5. The system of claim 1, wherein the one or more headset and one or more electronic device are in wired or wireless electronic communication with each other.

6. The system of claim 5, wherein the wireless electronic communication comprises BLUETOOTH®, Wi-Fi, 3G, LTE, 5G, or a wireless data communications network.

7. The system of claim 1, wherein the one or more electronic device is selected from the group consisting of: a mobile phone, a smart phone, a tablet, a tablet personal computer (PC), a PC, a server, a workstation, and a computer.

8. The system of claim 1, wherein the one or more electronic device is configured by the software application to display a graphical user interface (GUI) to the subject, wherein the one or more electronic device receives input from the subject via the GUI.

9. The system of claim 1, wherein the type of output comprises visual output, sound output, vibration output, or haptic output.

10. The system of claim 1, wherein analysis of the received electrical activity data comprises determining a baseline brain wave threshold by calculating an average brain wave power or amplitude value of the received electrical activity data during a baseline time interval.

11. The system of claim 10, wherein after the baseline time interval has concluded, the one or more electronic device is configured to continue receiving electrical activity data of the subject's brain;
wherein the average brain wave power or amplitude is an average alpha wave power or amplitude and the baseline brain wave threshold is a baseline alpha wave threshold.

12. The system of claim 11, wherein in response to the one or more electronic device receiving, after the baseline time interval, electrical activity data having an alpha power or amplitude greater than the baseline alpha wave threshold, the one or more electronic device is configured to transmit the output with an intensity set to a level indicating that the subject is acting in a manner that is consistent with a calmer brain state.

13. The system of claim 11, wherein in response to the one or more electronic device receiving, after the baseline time interval, electrical activity data having an alpha power or amplitude lower than the baseline alpha wave threshold, the one or more electronic device is configured to transmit the output with an intensity set to a level indicating that the subject is acting in a manner that is not consistent with a calmer brain state.

14. A method for reducing pain in a subject, the method comprising:
detecting, using a headset, electrical activity of the brain of the subject;
receiving, at one or more electronic device, electrical activity data of the subject's brain from the one or more headset;
analyzing, at the one or more electronic device, the received electrical activity data of the subjects brain;
transmitting an output to the subject, via an output electronic device, based on a result of analyzing the received electrical activity data of the subject's brain, where the output electronic device is the one or more electronic device or another device in communication with the one or more electronic device;
wherein the output is configured to reduce or relieve pain in the subject.

15. The method of claim 14, wherein the one or more headset comprises an electroencephalography (EEG) headset or headband.

16. The method of claim 14 further comprising continuously measuring EEG power or amplitude of brain waves of the subject while the headset is on the subject and powered on.

17. The method of claim 16, wherein the brain waves of the subject include brain waves selected from the group consisting of alpha waves, delta waves, theta waves, beta waves, and/or gamma waves.

18. The method of claim 14, wherein the one or more electronic device is selected from the group consisting of: a mobile phone, a smart phone, a tablet, a tablet personal computer (PC), a PC, a server, a workstation, and a computer.

19. The method of claim 14 further comprising:
displaying a graphical user interface (GUI) to the subject; and
receiving input from the subject via the GUI.

20. The method of claim 14, wherein the output comprises visual output, sound output, vibration output, or haptic output.

21. The method of claim 14, wherein analyzing the received electrical activity data comprises determining a baseline brain wave threshold by calculating an average brain wave power or amplitude value of the received electrical activity data during a baseline time interval.

22. The method of claim 21, wherein after the baseline time interval has concluded, the one or more electronic device is configured to continue receiving electrical activity data of the subject's brain,
wherein the average brain wave power or amplitude is an average alpha wave power or amplitude and the baseline brain wave threshold is a baseline alpha wave threshold.

23. The method of claim 22, responsive to the one or more electronic device receiving, after the baseline time interval, electrical activity data having an alpha power or amplitude greater than the baseline alpha wave threshold, the method further comprises transmitting, by the one or more electronic device, the output with an intensity set to a level indicating that the subject is acting in a manner that is consistent with a calmer brain state.

24. The method of claim 22, responsive to the one or more electronic device receiving, after the baseline time interval, electrical activity data having an alpha power or amplitude lower than the baseline alpha wave threshold, the method further comprises transmitting, by the one or more electronic device, the output with an intensity set to a level indicating that the subject is acting in a manner that is not consistent with a calmer brain state.

25. An electronic device comprising one or more processors configured to operate one or more software applications, which, when operated by the one or more processors, configure the one or more electronic device to perform the following functions:
receive electrical activity data corresponding to brain electrical activity of a subject; and
provide or transmit:
an output to the subject at an intensity, the intensity being based on an analysis of the received electrical activity data of the subject's brain; or
a control signal to an output electronic device to provide an output to the subject at an intensity based on an analysis of the received electrical activity data of the subject's brain;
wherein the output is configured to reduce or relieve pain in the subject.

26. A method for reducing pain in a subject, the method comprising:
receiving electrical activity data corresponding to brain electrical activity of a subject; and
providing or transmitting:
an output to the subject at an intensity, where the intensity is based on an analysis of the received electrical activity data of the subject's brain; or
a control signal to an output electronic device to provide an output to the subject at an intensity based on an analysis of the received electrical activity data of the subject's brain:
wherein the output is configured to reduce or relieve pain in the subject.
